(19) United States
(12) Patent Application Publication
(10) Pub. No.: US 2026/0108503 A1
(43) Pub. Date: Apr. 23, 2026

(54) POLYMERIC MICELLE NANOCARRIERS FOR TARGETED EPIDERMAL DELIVERY OF THE HEDGEHOG PATHWAY INHIBITOR TAK-441

(71) Applicant: UNIVERSITÉ DE GENÈ DE GENÈVE, Geneva (CH)

(72) Inventors: Aditya R. DARADE, Geneva (CH); Maria LAPTEVA, Geneva (CH); Yogeshvar N. KALIA, Geneva (CH)

(21) Appl. No.: 19/117,687

(22) PCT Filed: Oct. 3, 2023
(86) PCT No.: PCT/IB2023/000608

§ 371 Date: Apr. 2, 2025

Related U.S. Application Data
(60) Provisional application No. 63/413,066, filed on Oct. 4, 2022.

(51) Int. Cl. A61K 31/437; A61K 9/107; A61K 47/02; A61K 47/10; A61K 47/22; A61K 47/38

(52) U.S. Cl. CPC A61K 31/437; A61K 9/1075; A61K 47/02; A61K 47/10; A61K 47/22; A61K 47/38

(57) ABSTRACT

Micelle compositions comprising a hedgehog pathway inhibitor and their use in treating skin diseases, conditions, or disorders, such as skin cancers, including basal cell carcinoma, are disclosed.

### RELATED APPLICATION INFORMATION

This application is a national stage application under 35 U.S.C. § 371 of PCT/IB2023/000608, filed Oct. 3, 2023, which claims priority to U.S. Application No. 63/413,066 filed on Oct. 4, 2022, the contents of which are herein incorporated by reference.

### BACKGROUND

Basal cell carcinoma is one of the most common cancers in the world constituting approximately 90% of all skin cancers with an incidence of 100 in 100,000 in the UK and 884 in 100,000 in Australia (Madan et al., 2010; Staples et al., 2006). The major causative factor is UV light (Couvé-Privat et al., 2002; Daya-Grosjean and Sarasin, 2000). UV-B damage causes C to T (or CC to TT) structural mutations in the DNA of epidermal basal cells (Athar et al., 2006).

The hedgehog (HH) signaling pathway is prominently involved in the progression of BCC. It is highly active during embryonic development but is inactive in most adult tissues except to maintain stem cell populations and to regulate the growth of hair follicles and sebaceous glands (Athar et al., 2006). Mutations in the Patched 1 (PTCH1) and smoothened (SMO) proteins, however, lead to a loss of function in PTCH1 or gain of function in SMO. These changes in function, respectively, can lead to activation of the GLI family of transcription factors (Dlugosz et al., 2012), resulting in the hyperproliferation of basal cells seen in BCC (Roewert-Huber et al., 2007; Samarasinghe and Madan, 2012). Inactivation of PTCH1 has been proposed to be a necessary step in progression of BCC (Gailani and Bale, 1997).

Locally advanced BCC patients are not eligible for surgery or radiotherapy (Gould et al., 2014). As a result, pharmacotherapies involving the inhibition of SMO and hence preventing activation of the HH signaling pathway have been developed. Vismodegib is a “first-in-class” synthetic inhibitor of SMO (Robarge et al., 2009; Gould et al., 2014), which was approved by the U.S. Food and Drug administration (FDA) for the treatment of metastatic or locally advanced BCC in 2012 (Dlugosz et al., 2012). Sonidegib was another FDA-approved hedgehog inhibitor for the treatment of locally advanced BCC in 2015 (Burness, 2015).

Mutations in SMO, however, can inhibit its interaction with such drugs, resulting in resistance to the treatment. Even before the approval of vismodegib by the FDA in 2012, the first cases of acquired resistance to vismodegib treatment due to mutation in SMO (SMO-D473H) were reported in 2009 (Yauch et al., 2009). Treatment with sonidegib in patients with resistance to vismodegib was ineffective (Jain et al., 2017). Furthermore, resistance to treatment with sonidegib, due to mutations in the drug binding site of SMO (SMO-Q476 and SMO-D473), also has been reported (Danial et al., 2016; Jain et al., 2017; Nguyen and Cho, 2022).

### SUMMARY

In some aspects, the presently disclosed subject matter provides a composition comprising a hedgehog pathway inhibitor and a polymeric surfactant. In certain aspects, the hedgehog pathway inhibitor is active against vismodegib-resistant Smoothened receptor D473H mutant.

In certain aspects, the hedgehog pathway inhibitor is selected from TAK-441, Vismodegib, Saridegib/Patidegib, Glasdegib, Sonidegib, Taladegib (Env-101), and BMS-833923 (XL-139). In particular aspects, the hedgehog pathway inhibitor comprises TAK-441.

In certain aspects, the polymeric surfactant is biocompatible and/or biodegradable. In certain aspects, the polymeric surfactant is selected from D-α-Tocopherol polyethylene glycol 1000 succinate (TPGS), mPEG-dihex-PLA, a poloxamer, poly(e-caprolactone), a poly(L-amino acid), and polyvalerolactone. In particular aspects, the polymeric surfactant comprises D-α-Tocopherol polyethylene glycol 1000 succinate (TPGS).

In certain aspects, the composition comprising a hedgehog pathway inhibitor and a polymeric surfactant comprises a micelle composition. In certain aspects, the micelle composition comprises spherical micelles having a diameter with a range from about 10 nm to about 100 nm. In particular aspects, the micelle composition comprises spherical micelles having a diameter with a range from about 10 nm to about 15 nm.

In certain aspects, the composition comprises a concentration of TPGS having a range from about 5 mg/mL to about 300 mg/mL. In particular aspects, the composition comprises a concentration of TPGS of about 10 mg/mL.

In certain aspects, the composition comprises TAK-441 having a range from about 100 to about 500 mg of TAK-441 per gram of TPGS. In particular aspects, the concentration of TAK-441 has a range from about 100 to about 300 mg of TAK-441 per gram of TPGS.

In other aspects, the composition further comprises a hydrogel. In certain aspects, the hydrogel is selected from hydroxypropylcellulose (HPC), hydroxypropyl methylcellulose (HPMC), a cellulose-based gel forming agent, and a poloxamer-based gelling agent. In particular aspects, the hydrogel is selected from HPC and HPMC.

In particular aspects, the HPMC is selected from: (a) an HPMC having a molecular mass of about 26 kDa, a methoxyl content having a range between about 19% to about 24%, and a hydroxypropoxyl content having a range between about 7% to about 12%; (b) an HPMC having a molecular mass of about 10 kDa, a methoxyl content having a range between about 28% to about 30%, and a hydroxypropoxyl content between about 7% to about 12%; and (c) combinations thereof.

In more particular aspects, the composition comprises about 0.25% (w/w) TAK-441; about 0.93% (w/w) TPGS; about 5% (w/w) of an HPMC having a molecular mass of about 26 kDa, a methoxyl content having a range between about 19% to about 24%, and a hydroxypropoxyl content having a range between about 7% to about 12%; and about 3% of an HPMC having a molecular mass of about 10 kDa, a methoxyl content having a range between about 28% to about 30%, and a hydroxypropoxyl content between about 7% to about 12%.

In certain aspects, the composition further comprises a rheology modifier. In certain aspects, the rheology modifier is selected from glycerol, a low and high molecular weight cellulose, and a sorbitol. In particular aspects, the rheology modifier comprises glycerol.

In certain aspects, the composition further comprises a preservative. In certain aspects, the preservative is selected from sodium metabisulfite, benzyl alcohol, benzalkonium chloride, chlorobutanol, sodium benzoate, potassium sorbate, methylparaben and propylparaben. In particular aspects, the preservative comprises sodium metabisulfite.

In certain aspects, the composition retains between about 90% to 100% hedgehog pathway inhibitor content after storage for about 6 months.

In other aspects, the presently disclosed subject matter provides a method for treating a disease, disorder, or condition associated with a hedgehog (HH) signaling pathway, the method comprising administering to a subject in need of treatment thereof, the presently disclosed composition described hereinabove.

In certain aspects, the composition is administered topically. In particular aspects, the composition is delivered cutaneously. In particular aspects, the composition is delivered to a viable epidermis of the subject. In particular aspects, the composition is delivered to an upper dermis of the subject. In particular aspects, the topical administration results in negligible transdermal permeation.

In certain aspects, the disease, disorder, or condition associated with a hedgehog (HH) signaling pathway comprises a skin disease, disorder, or condition. In particular aspects, the skin disease, disorder, or condition comprises a skin cancer. In more particular aspects, the skin cancer comprises basal cell carcinoma. In yet more particular aspects, the subject has or is suspected of having locally advanced basal cell carcinoma. In yet more particular aspects, the subject has or is suspected of having metastatic basal cell carcinoma. In certain aspects, the subject is not eligible for surgical or radiotherapeutic treatment. In more certain aspects, administering the composition to the subject attenuates progression of the basal cell carcinoma. In particular aspects, the basal cell carcinoma involves a vismodegib-resistant SMO mutant D473H.

In certain aspects, administering the composition to the subject prevents activation of the HH signaling pathway.

In certain aspects, the disease, condition, or disorder involves a mutation in a Patched 1 (PTCH1) protein. In particular aspects, the mutation in the Patched 1 (PTCH1) protein leads to a loss of function of the PTCH1 protein. In certain aspects, the disease, condition, or disorder involves a mutation in a smoothened (SMO) protein. In particular aspects, the mutation in the smoothened (SMO) protein involves a gain of function in the SMO protein. In more particular aspects, the loss of function of the PTCH1 protein or the gain of function in the SMO protein leads to activation of one or more GLI transcription factors. In yet more particular aspects, the activation of one or more GLI transcription factors results in hyperproliferation of basal cells associated with basal cell carcinoma.

In certain aspects, administering the composition to the subject inhibits SMO. In certain aspects, the mutation in SMO results in resistance to treatment with the hedgehog pathway inhibitor. In particular aspects, the resistance to treatment with the hedgehog pathway inhibitor involves a mutation in a drug binding site of SMO. In more particular aspects, the drug binding site of SMO is SMO-Q476 and/or SMO-D473.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 shows the chemical structure of TAK-441 (MW 576.57 Da; log P 2.61; aqueous solubility 81 μg/mL at pH 6.8) (Ohashi et al., 2012; Ishii et al., 2014);

FIG. 2 is a TEM image of an embodiment of the presently disclosed TAK-441 micelle formulation (3 mg/mL);

FIG. 3 is a rheogram of an embodiment of the presently disclosed TAK-441 loaded TPGS micelle-based 3% HPC gel;

FIG. 4A, FIG. 4B, and FIG. 4C show porcine skin deposition and biodistribution of TAK-441 (micelle solution and micelle HPC gel formulation, n=6). (FIG. 4A) Porcine skin deposition of TAK-441; (FIG. 4B) Porcine skin biodistribution of TAK-441 at infinite dose; and (FIG. 4C) Porcine skin biodistribution of TAK-441 at finite dose; (** P<0.05, one-way ANOVA). (Mean±SD);

FIG. 5 is a rheogram of an embodiment of the presently disclosed TAK-441 loaded TPGS micelle-based 3% HPMC gel;

FIG. 6 shows the stability of TAK-441 loaded micelle formulations. The TAK-441 content in Formulation E and the HPMC gel of Formulation E packaged in aluminum tubes (Nussbaum Kesswil AG, Switzerland) was quantified for 6 months (stored at 4° C.) using UHPLC-MS/MS at different time points. After 6 months, TAK-441 content in the micelle solution was 79.62% of the initial value, whereas in the micelle gel, TAK-441 content was 91.86% of the initial amount. The micelles were found to be intact in the gel formulation; and

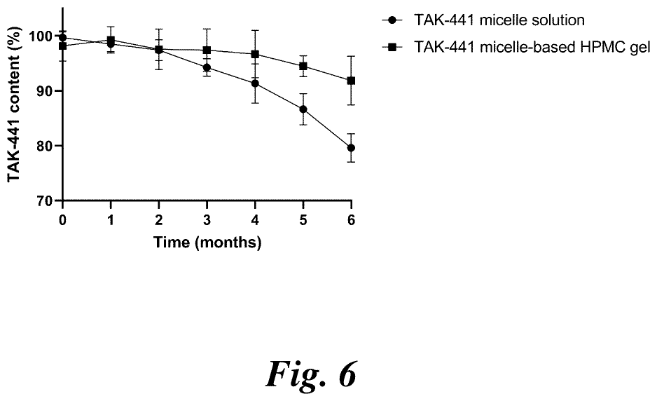

FIG. 7A, FIG. 7B, and FIG. 7C show human skin deposition and biodistribution of TAK-441 (micelle-based HPMC gel formulation, n=6). (FIG. 7A) Human skin deposition of TAK-441; (FIG. 7B) Human skin biodistribution of TAK-441 at infinite dose; and (FIG. 7C) Human skin biodistribution of TAK-441 at finite dose (** P<0.05, one way ANOVA). (Mean±SD).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

More particularly, in some embodiments, the presently disclosed subject matter provides a composition comprising a hedgehog pathway inhibitor and a polymeric surfactant. Representative hedgehog pathway inhibitors include, but are not limited to, vismodegib, sonidegib, and other hedgehog pathway inhibitors, in particular SMO inhibitors, currently under clinical trial include, but are not limited to, IPI-926 (saridegib), BMS-833923/XL139, PF-04449913 (glasdegib), and LY2940680 (taladegib). In certain embodiments, the hedgehog pathway inhibitor is active against vismodegib-resistant Smoothened receptor D473H mutant.

In certain embodiments, the hedgehog pathway inhibitor is selected from TAK-441, Vismodegib, Saridegib/Patidegib, Glasdegib, Sonidegib, Taladegib (Env-101), and BMS-833923 (XL-139). In particular embodiments, the hedgehog pathway inhibitor comprises TAK-441.

In certain embodiments, the polymeric surfactant is biocompatible and/or biodegradable. In certain embodiments, the polymeric surfactant is selected from D-α-Tocopherol polyethylene glycol 1000 succinate (TPGS), mPEG-dihex-PLA, a poloxamer, poly(e-caprolactone), a poly(L-amino acid), and polyvalerolactone. In particular embodiments, the polymeric surfactant comprises D-α-Tocopherol polyethylene glycol 1000 succinate (TPGS).

In certain embodiments, the composition comprising a hedgehog pathway inhibitor and a polymeric surfactant comprises a micelle composition.

As used herein, the term “micelle” refers to an aggregate of surfactant molecules. Micelles only form when the concentration of surfactant is greater than the critical micelle concentration (CMC). Surfactants are chemicals that are amphipathic, that is, they contain both hydrophobic and hydrophilic groups. Micelles can exist in different shapes, including spherical, cylindrical, and discoidal.

In certain embodiments, the micelle composition comprises spherical micelles having a diameter with a range from about 10 nm to about 100 nm, including 10, 15, 20, 25, 30, 35, 40, 45, 50, 55, 60, 65, 70, 75, 80, 85, 90, 95, and 100 nm In particular embodiments, the micelle composition comprises spherical micelles having a diameter with a range from about 10 nm to about 15 nm, including about 10, 11, 12, 13, 14, and 15 nm.

Polymeric micelles can be used as nanocarriers for the delivery of poorly water-soluble, water-insoluble, or hydrophobic drugs, which can be solubilized in the hydrophobic inner core of a micelle. Micelles can therefore serve to improve solubility and bioavailability of various hydrophobic drugs. The small size of micelles (typically about 10 nm to about 100 nm, including about 10, 15, 20, 25, 30, 35, 40, 45, 50, 55, 60, 65, 70, 75, 80, 85, 90, 95, and 100 nm) allows for efficient accumulation of an associated active moiety into targeted tissues. Micelles can be formed from one or more polymeric nonionic surfactants.

As provided hereinabove, in particular embodiments, the surfactant comprises a tocopherol or derivative thereof. Tocopherols are a class of methylated phenols, many of which have vitamin E activity. Tocopherols and their derivatives, such as esters for example, are widely used in vitamin supplementation and as antioxidants in the food industry and in many pharmaceutical compositions. Tocopherols include a range of natural and synthetic compounds. Vitamin E α-Tocopherol (chemical name: 2,5,7,8-tetramethyl-2-(4′,8′, 12′-trimethyldecyl)-6-chromanole) is the most active and widely distributed in nature, and has been the most widely studied. Other members of the class include beta, gamma, and delta tocopherols. Tocopherols occur in a number of isomeric forms, the D and DL forms being the most widely available. As used herein, the term “tocopherol” includes all such natural and synthetic tocopherol or Vitamin E compounds.

Any of the forms or isomers of tocopherols and their derivatives, e.g., esters, can be used according to the present disclosure. For example, α-tocopherol or its esters including, but not limited to, α-tocopherol acetate, linoleate, nicotinate or hemi succinate-ester, many of which are available commercially, can be used herein.

The tocopherol derivatives include chemical derivatives of vitamin E with ester and ether linkages of various chemical moieties to polyethylene glycol of various lengths. For example, the derivative may include vitamin E tocopherol polyethylene glycol succinate (TPGS) derivatives with PEG molecular weights between about 500 and 6000 Da, including about 500, 1000, 1500, 2000, 2500, 3000, 3500, 4000, 4500, 5000, 5500, and 6000 Da. In particular embodiments, the vitamin E polymeric derivative is D-α-Tocopherol polyethylene glycol 1000 succinate (TPGS).

More particularly, TPGS is a water soluble derivative of Vitamin E in which polyethylene glycol subunits are attached by a succinic acid diester at the ring hydroxyl of the vitamin E molecule. TPGS is an almost odorless waxy amphiphilic substance with a molecular weight about 1513. TPGS forms stable micelles in aqueous vehicles for its amphiphilic structure with a hydrophile/lipophile balance (HLB) value of 13.2. TPGS has been approved as a pharmaceutical excipient by the U.S. Food and Drug Administration (FDA).

The tocopherol surfactant of the disclosure may be used alone or in combination with other known surfactants, e.g., phospholipids, polysorbates, sorbitan esters of fatty acids, cetearyl glucoside or poloxamers, or other stabilizers, such as xanthan gum or propylene glycol alginate.

In certain embodiments, the composition comprises a concentration of TPGS having a range from about 5 mg/mL to about 300 mg/mL, including about 5, 10, 15, 20, 25, 30, 35, 40, 45, 50, 55, 60, 65, 70, 75, 80, 85, 90, 100, 110, 120, 130, 140, 150, 160, 170, 180, 190, 200, 210, 220, 230, 240, 250, 260, 270, 280, 290, and 300 mg/mL. In particular embodiments, the composition comprises a concentration of TPGS of about 10 mg/mL.

In certain embodiments, the composition comprises TAK-441 having a range from about 100 to about 500 mg of TAK-441 per gram of TPGS, including about 100, 125, 150, 175, 200, 225, 250, 275, 300, 325, 350, 375, 400, 425, 450, 475, and 500 mg. In particular embodiments, the concentration of TAK-441 has a range from about 100 to about 300 mg of TAK-441 per gram of TPGS.

In other embodiments, the composition further comprises a hydrogel. In certain embodiments, the hydrogel is selected from hydroxypropylcellulose (HPC), hydroxypropyl methylcellulose (HPMC), a cellulose-based gel forming agent, and a poloxamer-based gelling agent. In particular embodiments, the hydrogel is selected from HPC and HPMC.

In particular embodiments, the HPMC is selected from: (a) an HPMC having a molecular mass of about 26 kDa, a methoxyl content having a range between about 19% to about 24%, including 19, 20, 21, 22, 23, and 24%, and a hydroxypropoxyl content having a range between about 7% to about 12%, including about 7, 8, 9, 10, 11, and 12%; (b) an HPMC having a molecular mass of about 10 kDa, a methoxyl content having a range between about 28% to about 30%, including 28, 29, and 30%, and a hydroxypropoxyl content between about 7% to about 12%, including about 7, 8, 9, 10, 11, and 12%; and (c) combinations thereof.

In more particular embodiments, the composition comprises about 0.25% (w/w) TAK-441; about 0.93% (w/w) TPGS; about 5% (w/w) of an HPMC having a molecular mass of about 26 kDa, a methoxyl content having a range between about 19% to about 24%, and a hydroxypropoxyl content having a range between about 7% to about 12%; and about 3% of an HPMC having a molecular mass of about 10 kDa, a methoxyl content having a range between about 28% to about 30%, and a hydroxypropoxyl content between about 7% to about 12%.

In certain embodiments, the composition further comprises a rheology modifier. In certain embodiments, the rheology modifier is selected from glycerol, a low and high molecular weight cellulose, and a sorbitol. In particular embodiments, the rheology modifier comprises glycerol.

In certain embodiments, the composition further comprises a preservative. In certain embodiments, the preservative is selected from sodium metabisulfite, benzyl alcohol, benzalkonium chloride, chlorobutanol, sodium benzoate, potassium sorbate, methylparaben and propylparaben. In particular embodiments, the preservative comprises sodium metabisulfite. Exemplary preservatives further include, but are not limited to, sorbic acid, benzoic acid, methyl-paraben, propyl-paraben, methylchloroisothiazolinone, methylisothiazolinone, diazolidinyl urea, chlorobutanol, triclosan, benzethonium chloride, p-hydroxybenzoate, chlorhexidine, digluconate, hexadecyltrimethyl ammonium bromide, alcohols, benzalkonium chloride, boric acid, bronopol, butylparaben, butylene calcium acetate, calcium chloride, calcium lactate, carbon dioxide, cationic, bentonite, cetrimide, cetylpyridinium chloride, chlorhexidine, chlorobutanol, chlorocresol, chloroxylenol, citric acid monohydrate, cresol, dimethyl ether, ethylparaben, glycerin, hexetidine, imidurea, isopropyl alcohol, lactic acid, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium sorbate, propionic acid, propyl gallate, propylene glycol, sodium acetate, sodium benzoate, sodium borate, sodium lactate, sodium sulfite, sodium propionate, xylitol, sulphur dioxide, carbon dioxide, and combinations thereof.

In certain embodiments, the composition retains between about 90% to 100% hedgehog pathway inhibitor content, including about 90, 91, 92, 93, 94, 95, 96, 97, 98, 99, and 100%, after storage for about 6 months, including 1, 2, 3, 4, 5, 6, 7, 8, and 9 months.

In other embodiments, the presently disclosed subject matter provides a method for treating a disease, disorder, or condition associated with a hedgehog (HH) signaling pathway, the method comprising administering to a subject in need of treatment thereof, the presently disclosed composition described hereinabove.

In certain embodiments, the composition is administered topically. In particular embodiments, the composition is delivered cutaneously, i.e., pertaining to the skin. In particular embodiments, the composition is delivered to a viable epidermis, i.e., the layer of skin immediately below the stratum corneum, of the subject. In particular embodiments, the composition is delivered to an upper dermis of the subject. The dermis includes the papillary dermis, the uppermost layer of the dermis, and the reticular dermis, the lower layer of the dermis, found under the papillary dermis. In particular embodiments, the topical administration results in negligible transdermal permeation. Generally, transdermal permeation includes penetration of the therapeutic agent through the stratum corneum and passing through the deeper epidermis and dermis without drug accumulation in the dermal layer. Negligible transdermal permeation can include about 0.001%, 0.01%, 0.1%, and 1% drug accumulation in the dermal layer.

In certain embodiments, the disease, disorder, or condition associated with a hedgehog (HH) signaling pathway comprises a skin disease, disorder, or condition. In particular embodiments, the skin disease, disorder, or condition comprises a skin cancer. In more particular embodiments, the skin cancer comprises basal cell carcinoma. In yet more particular embodiments, the subject has or is suspected of having locally advanced basal cell carcinoma. In yet more particular embodiments, the subject has or is suspected of having metastatic basal cell carcinoma. In certain embodiments, the subject is not eligible for surgical or radiotherapeutic treatment. In more certain embodiments, administering the composition to the subject attenuates progression of the basal cell carcinoma. In particular embodiments, the basal cell carcinoma involves a vismodegib-resistant SMO mutant D473H.

In certain embodiments, administering the composition to the subject prevents activation of the HH signaling pathway.

In certain embodiments, the disease, condition, or disorder involves a mutation in a Patched 1 (PTCH1) protein. In particular embodiments, the mutation in the Patched 1 (PTCH1) protein leads to a loss of function of the PTCH1 protein. In certain embodiments, the disease, condition, or disorder involves a mutation in a smoothened (SMO) protein. In particular embodiments, the mutation in the smoothened (SMO) protein involves a gain of function in the SMO protein. In more particular embodiments, the loss of function of the PTCH1 protein or the gain of function in the SMO protein leads to activation of one or more GLI transcription factors. In yet more particular embodiments, the activation of one or more GLI transcription factors results in hyperproliferation of basal cells associated with basal cell carcinoma.

In certain embodiments, administering the composition to the subject inhibits SMO. In certain embodiments, the mutation in SMO results in resistance to treatment with the hedgehog pathway inhibitor. In particular embodiments, the resistance to treatment with the hedgehog pathway inhibitor involves a mutation in a drug binding site of SMO. In more particular embodiments, the drug binding site of SMO is SMO-Q476 and/or SMO-D473.

The presently disclosed composition can be administered as a monotherapy or in combination with other therapies, including photodynamic therapy (PDT).

The term “combination” is used in its broadest sense and means that a subject is administered at least two agents or therapies, such as the presently disclosed composition and at least one other therapeutic agent or therapy. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the presently disclosed compositions can be administered alone or in combination with adjuvants that enhance stability of the composition, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a presently disclosed composition and at least one additional therapeutic agent or therapy can be varied so long as the beneficial effects of the combination of these agents are achieved. Accordingly, the phrase “in combination with” refers to the administration of a composition described herein and at least one additional therapeutic agent or therapy either simultaneously, sequentially, or a combination thereof. Therefore, a subject administered a combination of a composition described herein and at least one additional therapeutic agent or therapy can receive a presently disclosed composition and at least one additional therapeutic agent or therapy at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound described herein and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + B / {Q}_{B} = Synergy Index ( SI )$$

wherein:
   Q_{A} is the concentration of a component A, acting alone, which produced an end point in relation to component A;
   Qₐ is the concentration of component A, in a mixture, which produced an end point;
   Q_{B} is the concentration of a component B, acting alone, which produced an end point in relation to component B; and
   Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

As used herein, the term “treating” can include reversing, alleviating, inhibiting the progression of, preventing, or reducing the likelihood of the disease, disorder, or condition to which such term applies, or one or more symptoms or manifestations of such disease, disorder, or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent refers to the amount necessary to elicit the desired biological response. As will be appreciated by those of ordinary skill in this art, the effective amount of an agent may vary depending on such factors as the desired biological endpoint, the agent to be delivered, the makeup of the pharmaceutical composition, the drug target, and the like.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Formulation Development and Cutaneous Biodistribution in Porcine and Human Skin

### 1.1 Overview

TAK-441 is a potent inhibitor of the hedgehog pathway (IC₅₀ 4.4 nM), active against vismodegib-resistant Smoothened receptor D473H mutant, and intended for the treatment of basal cell carcinoma. This example describes the development of a micelle-based formulation of TAK-441 using D-α-Tocopherol polyethylene glycol 1000 succinate (TPGS) and the investigation its cutaneous delivery and biodistribution. The results demonstrated that incorporation of TAK-441 in TPGS micelles increased aqueous solubility approximately 40-fold. One embodiment, an HPMC hydrogel of TAK-441 loaded TPGS micelles, retained approximately 92% of the initial TAK-441 content after storage at 4° C. for 6 months. Finite dose experiments using human skin demonstrated that this formulation resulted in significantly greater cutaneous deposition of TAK-441 after 12 h than a non-micelle control formulation, (0.40±0.11 μg/cm² and 0.05±0.02 μg/cm², respectively). Further, no transdermal permeation was observed. The cutaneous biodistribution profile demonstrated that TAK-441 was predominantly delivered to the viable epidermis and upper dermis. Delivery from the HPMC hydrogel formulation resulted in TAK-441 epidermal concentrations that were several thousand-fold higher than the IC₅₀, with almost negligible transdermal permeation, thereby decreasing the risk of systemic side effects in vivo.

### 1.2 Background

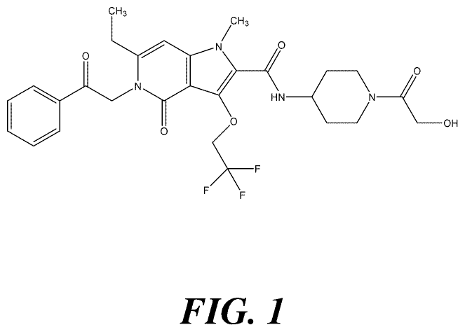

TAK-441 is a potent inhibitor of the HH pathway (IC₅₀ 4.4 nM; determined by luciferase reporter activities in NIH3T3 cells carrying a stably transfected Gli-reporter construct) and is effective against vismodegib-resistant SMO mutant D473H (Goldman et al., 2015; Ishii et al., 2014; Ohashi et al., 2012). Ishii et al. reported TAK-441 as having an IC₅₀ of 79 nM in D473H-transfected cells; in comparison, the IC₅₀ of vismodegib was 7100 nM. TAK-441 has a molecular weight of 576.57 Da (FIG. 1), is moderately lipophilic (log P 2.61) but has very poor aqueous solubility (81 μg/mL at pH 6.8) (Ohashi et al., 2012; Ishii et al., 2014). Phase I clinical trials have investigated its oral administration at doses ranging from 50 mg/day up to a maximum feasible dose (MFD) of 1600 mg/day (Goldman et al., 2015). Gli1 expression in skin biopsies was strongly inhibited at all doses, but there were various side effects. All patients experienced at least one adverse effect (AE); mild to moderate AE were dysgeusia, fatigue, nausea, muscle spasm, and hyponatremia (Goldman et al., 2015). Approximately 35% of patients experienced serious AE including gastrointestinal disorders, neoplasms (progression of underlying disease), and hepatobiliary disorders. One death due cerebral hemorrhage in a patient with pancreatic cancer was assessed to be study drug-related by the investigator.

Topical delivery of TAK-441 could not only improve the efficacy through better targeting the site of disease but also increase treatment tolerability by reducing systemic side effects. Direct application to the disease site obviously lowers the dose required as compared to oral administration and by definition reduces systemic or “off-target” toxicity. Topical pharmacotherapy using TAK-441, however, must ensure sufficient cutaneous bioavailability and more specifically that supra-therapeutic concentrations are achieved in the basal epidermis. Given its poor aqueous solubility, it is simpler to formulate TAK-441 in a more lipophilic system where it is more soluble; however, that greater solubility and hence formulation stability comes at the expense of a lower thermodynamic activity and less favorable partitioning into the stratum corneum.

Polymeric micelles are colloidal nanocarriers formed of polymeric surfactants that self-assemble in aqueous media at concentrations above the critical micelle concentration (Lavasanifar et al., 2002). We have previously described how methoxypoly(ethylene glycol)-di-hexyl-substituted-poly(lactic acid) (mPEGhexPLA) micelles can be used to develop aqueous formulations of several poorly water soluble therapeutics with dermatological applications: econazole (Bachhav et al., 2011), tacrolimus (Lapteva et al., 2014a), ciclosporin (Lapteva et al., 2014b), retinoic acid (Lapteva et al., 2015), imiquimod (Lapteva et al., 2019) and spironolactone (Dahmana et al., 2021) and enable their improved cutaneous delivery as compared to existing approved formulations (Lapteva et al., 2014a, 2015, 2019). Furthermore, in another study, mPEGhexPLA micelles were used to develop the first topical formulation of vismodegib and the cutaneous biodistribution method used to show that therapeutically relevant amounts of drug could be delivered into the epidermis and upper dermis (Kandekar et al., 2019).

In other studies, we have used D-α-tocopherol polyethylene glycol succinate 1000 (TPGS) as the copolymer (Kandekar et al., 2018), sirolimus (Quartier et al., 2021a) and co-formulation of econazole, terbinafine and amorolfine (Gou et al., 2022). This biocompatible and biodegradable surfactant is an amphiphilic derivative of natural vitamin E and has been approved by the regulatory authorities as an excipient for pharmaceutical products (Aggarwal et al., 2012). TPGS has been approved by the FDA as a pharmaceutical ingredient and has been used as an excipient in various marketed products (Zhang et al., 2015; Vadlapudi et al., 2014). TPGS also was approved as an active pharmaceutical ingredient (API) by the European Medicines Agency on Jul. 24, 2009 (Vendrop®), for the treatment of vitamin E deficiency due to digestive malabsorption in pediatric patients suffering from congenital chronic cholestasis or hereditary chronic cholestasis (Papas, 2021).

### 1.3 Scope

The objectives of this example were (i) to investigate the feasibility of using TPGS micelles to overcome the intrinsic poor aqueous solubility of TAK-441 and to develop a stable aqueous formulation; (ii) to characterize the micelles in terms of drug content, size, and morphology; (iii) to develop a user-friendly micelle-based hydrogel formulation for topical application; (iv) to study the cutaneous delivery of TAK-441 and to determine the cutaneous biodistribution in porcine skin after application of micelle solution and micelle-based hydrogel formulations and to compare the results to those obtained with a non-micelle control formulation; and (v) to confirm the results using human skin.

### 1.4 Materials and Methods

### 1.4.1 Materials

TAK-441 was kindly provided by Takeda Pharmaceutical Company Ltd, Japan. D-α-Tocopherol polyethylene glycol 1000 succinate (TPGS), formic acid (MS grade), isopentane, and Dulbecco's phosphate-buffered saline (DPBS), hydroxypropyl methylcellulose (HPMC, approximately 26 kDa; methoxyl content 19-24% and hydroxypropoxyl content 7-12%) were purchased from Sigma-Aldrich (Buchs, Switzerland). Low molecular weight HPMC—Methocel™ E5 Premium LV (approximately 10 kDa; methoxyl content 28-30% and hydroxypropoxyl content 7-12%) was procured from Dow chemicals (Horgen, Switzerland). Hydroxypropyl cellulose (Klucel™ MF Pharm, HPC; M.W. approximately 850 kDa) and glycerol were purchased from Hänseler AG (Herisau, Switzerland). Bovine serum albumin (BSA) was purchased from Axon Lab (Baden-Dättwil, Switzerland). Acetone (analytical grade) and Nile-Red dye were obtained from Acros Organics (Geel, Belgium). Methanol and acetonitrile (LC-MS grade) were purchased from Fisher Scientific (Reinach, Switzerland). PTFE membrane filters (0.22 μm), Amicon Ultra 0.5 mL (5 kDa) filtration units were purchased from VWR (Nyon, Switzerland). Ultrapure water (Millipore Milli-Q Gard 1 Purification Pack resistivity >18 MΩcm; Zug, Switzerland) was used for formulation development and analysis. All other chemicals were at least of analytical grade.

### 1.4.2 Analytical Methods

TAK-441 was quantified using a Waters Acquity Core UHPLC® system equipped with Xevo® TQ-MS tandem quadrupole detector. Isocratic separation was performed using an Acquity UHPLC® BEH C18 column (2.1×50 mm; 1.7 μm) in tandem with an Acquity UHPLC® C18 VanGuard pre-column (2.1×5 mm, 1.7 μm) that was maintained at 25° C. The mobile phase consisted of a mixture of acetonitrile and water (75:25 v/v). The flow rate and injection volume were 0.1 mL/min and 5 μL, respectively. A peak for TAK-441 was obtained at 1.7 min, and the run time was 3.0 min. Mass spectrometric detection was performed with electrospray ionization in positive ion mode using multiple reaction monitoring (MRM). The detection settings for TAK-441 are presented in Table 1. The limits of detection (LOD) and quantification (LOQ) were 1.29 and 3.29 ng/mL, respectively. The UHPLC-MS/MS method was validated as per ICH guidelines.

TABLE 1
MS/MS Settings for the detection of TAK-441

| | TAK-441 |
|---|---|
| Nature of parent ion | Hydrogen adduct [M + H]⁺ |
| Parent ion (m/z) | 577.40 |
| Daughter ions (m/z) | 419.26 |
| Collision energy (V) | 32 |
| Cone voltage (V) | 36 |
| Capillary voltage (kV) | 2.9 |
| Desolvation temperature (° C.) | 350 |
| Desolvation gas flow (L/h) | 650 |
| Cone gas flow (L/h) | 2 |
| Collision gas flow (mL/min) | 0.15 |
| LM resolution 1 | 2.96 |
| HM resolution 1 | 15 |
| Ion energy 1 (V) | 0.3 |
| LM resolution 2 | 2.91 |
| HM resolution 2 | 15.24 |
| Ion energy 2 (V) | 0.6 |

### 1.4.3 Preparation of Micelle Formulations

### 1.4.3.1 Micelle Solution

TPGS based micelles of TAK-441 were prepared by the solvent evaporation method (Kandekar et al., 2019). The screening of surfactants and their concentration was done using a micro-scale formulation technique, which includes simultaneous multiple experimentation with minimum amounts of drug and excipients. This process reduces the material cost, time required for excipient screening and formulation development, as well as decreasing exposure to the drug, which is beneficial when working with cytotoxics.

Short-listed formulations were then scaled up to lab-scale batches. Briefly, the known amounts of TPGS and TAK-441 were dissolved in 2 mL of acetone to obtain a clear solution. This solution was added slowly to 4 mL water under sonication (Branson Digital Sonifier S-450D). Acetone was then slowly removed by using a rotary evaporator (Büchi RE 121 Rotavapor). The final volume was made up with water in a volumetric flask to obtain the micelle formulation with TAK-441 and TPGS concentrations of 3 mg/mL and 10 mg/mL, respectively. After equilibration overnight, the micelle solution was centrifuged at 10,000 rpm for 15 min (Eppendorf Centrifuge 5804) to remove excess TAK-441, and the supernatant was carefully collected.

### 1.4.3.2 Micelle Gels

In preliminary studies, TAK-441-TPGS micelles were incorporated into 3% HPC gel to study the cutaneous delivery of TAK-441 from a semi-solid gel formulation. The formulation was compared with the control gel having the same composition except for the polymeric surfactant. Based on the preliminary results, it was decided to prepare a micelle-based HPMC gel with better formulation properties for clinical application (see below for complete details; section 1.5.1).

### 1.4.4 Characterization of Micelle Formulations

### 1.4.4.1 Size Determination

The hydrodynamic diameter (Zₐᵥ), polydispersity index (P.D.I.), and volume weighted and number weighted diameters (dᵥ and dₙ, respectively) of the micelles were measured using dynamic light scattering (DLS) with a Zetasizer HS 3000 (Malvern Instruments Ltd.; Malvern, UK). Measurements were performed at an angle of 90° and a temperature of 25° C. All values were obtained after 3 runs of 10 measurements.

### 1.4.4.2 Morphology

Micelle morphology was characterized with transmission electron microscopy (TEM) (FEI Tecnai G2 Sphera, Eindhoven, Netherlands) using the negative staining method. Briefly, 5 μL of the micelle solution was dropped onto an ionized carbon-coated copper grid (0.3 Torr, 400 V for 20 s). The grid was then placed for 1 s in a 100-μL drop of a saturated uranyl acetate aqueous solution and then in a second 100-μL drop for 30 s. The excess staining solution was removed, and the grid was dried at room temperature before the measurement.

### 1.4.4.3 Determination of TAK-441 Content in the Micelles

TAK-441 loaded into the micelles was quantified by UHPLC-MS/MS. To ensure complete micelle destruction and release of the incorporated drug, formulations were diluted in acetonitrile and analyzed. The drug content, drug loading, and entrapment efficiency were calculated using equations 1-3:

$$Drug content ( \frac{mg}{mL} ) = \frac{Drug in the formulation ( mg )}{Volume of the formulation ( mL )} \qquad ( 1 )$$

$$Drug loading ( \frac{mg of drug}{g of polymer} ) = \frac{Drug in the formulation ( mg / mL )}{Polymer in the formulation ( g / mL )} \qquad ( 2 )$$

$$Entrapment efficiency ( \% ) = \frac{Drug entrapped in micelles ( mg )}{Drug added ( mg )} \times 100 \qquad ( 3 )$$

### 1.4.4.4 Viscosity Measurements

The viscosity of micelle gels was measured by using a Thermo Scientific™ HAAKE™ MARS™ rheometer. The measurements were carried out at a fixed temperature (25° C.) using a rotating plate spindle at different shear rates. The measurements and post-measurement evaluations were carried out using Thermo Scientific™ HAAKE™ RheoWin software.

### 1.4.4.5 Evaluation of Micelle Formulation Stability

The TAK-441 micelle aqueous formulation and the micelle-based HPMC gel formulation were prepared and stored at 4° C. for 6 months. The formulations were assayed to determine drug content at various time points (day 1, followed by every month).

### 1.4.5 Cutaneous Delivery and Biodistribution Studies In Vitro

### 1.4.5.1 Skin Preparation

Porcine ears were purchased from a local abattoir (CARRE; Rolle, Switzerland) shortly after sacrifice. After washing under running cold water, skin samples with a thickness of approximately 0.8 mm were carefully harvested from the outer region of the ear using a Zimmer air dermatome (Münsingen, Switzerland). Hair was removed from the skin surface using clippers. Discs corresponding to the permeation area were punched out (Berg & Schmid HK 500; Urdorf, Switzerland). Skin samples were frozen at −20° C. and stored for a maximum period of 3 months. Before the experiment, skin samples were thawed at room temperature and placed for 15 min in 0.9% saline solution for rehydration.

Human skin samples were obtained shortly after surgery from the Department of Plastic, Aesthetic and Reconstructive Surgery, Geneva University Hospital (Geneva, Switzerland), fatty tissue was removed and the skin was stored at −20° C. The donation was approved by the Central Committee for Ethics in Research (CER: 08-150 (NAC08-051); Geneva University Hospital).

### 1.4.5.2 Micelle Solution

The experiments were performed using standard two-compartment vertical (Franz-type) diffusion cells, (Milian SA; Meyrin, Switzerland) with a cross-sectional area of 2 cm². The receptor compartment consisted of 10-mL Dulbecco's phosphate-buffered saline (DPBS) pH 7.4 containing 1% BSA to maintain sink conditions. The receiver compartment was maintained between 32° C.-34° C. For infinite dose conditions, 200 μL of TAK-441 micelle formulation (3 mg/mL) was applied to the skin sample surface (i.e., 300 μg of TAK-441/cm² of the skin surface) and for finite dose, 20 μL of micelle formulation (3 mg/mL) was applied (30 μg of TAK-441/cm² of the skin surface). A non-micelle formulation comprised of 3 mg/mL TAK-441 suspended in aqueous 0.05% hydroxypropyl cellulose (HPC) was used as a control.

TAK-441 had the least solubility in HPC; this characteristic would minimize the risk of interference of the suspending agent on drug delivery. Aliquots (1 mL) were withdrawn from the receiver compartment at 1 h, 4 h, and 12 h and replaced with an equivalent volume of fresh media. Samples were diluted in acetonitrile to precipitate BSA. After centrifugation at 10,000 rpm for 15 min, the permeation samples were analyzed by UHPLC-MS/MS.

Upon completion of the experiments, the excess formulation was removed from the skin surface using a validated wash method. The skin samples were cut into small pieces, and deposited TAK-441 was extracted by soaking the pieces in 2 mL of methanol for 4 h with continuous stirring at room temperature. The extraction procedure was validated. The extraction samples were centrifuged at 10,000 rpm for 15 min, diluted, and filtered through a 0.22-μm PTFE filter before UHPLC-MS/MS analysis.

### 1.4.5.3 Micelle Gel

TAK-441 micelles were incorporated into 3% HPC gel to test the cutaneous delivery of TAK-441 to porcine skin from a semi-solid gel formulation. The composition of the control gel was the same except for the polymeric surfactant. The experiments were performed as described above (section 1.4.5.2). For infinite dose, 200 mg of micelle gel (2.88 mg_{TAK-441}/g of gel formulation; i.e., the gel contained 0.29% TAK-441) was applied on the skin surface (i.e., 288 μg TAK-441/cm² of the skin surface) and for finite dose, 20 mg of micelle gel was applied (28.8 μg/cm² TAK-441 of the skin surface).

Similar experimental conditions were used for the micelle-based HPMC gel, which was used to test delivery to human skin. For infinite dose, 200 mg of micelle gel (2.5 mg_{TAK-441}/g of gel formulation; i.e., the gel contained 0.25% TAK-441) was applied on the skin surface (i.e., 250 μg TAK-441/cm² of the skin surface) and for finite dose, 20 mg of micelle gel was applied (25 μg/cm² TAK-441 of the skin surface). The composition of the control gel was the same as the HPMC gel except for the TPGS.

Upon completion of the experiment, a punch was used to separate the skin sample into two parts—an inner disk with a surface area of 0.785 cm² and a remaining outer ring with an area of 1.215 cm². This outer ring was subsequently cut into small pieces, and TAK-441 deposited in the tissue was extracted by the validated extraction method (section 1.4.5.2) followed by quantification using UHPLC-MS/MS.

The 0.785-cm² disks were used to determine the TAK-441 biodistribution as a function of depth in the skin. These skin discs were snap-frozen in isopentane cooled by liquid nitrogen. For this procedure, the skin samples were fixed with O.C.T. on a circular piece of cork and a plastic O-ring was placed around the skin discs to avoid tissue compression and to ensure a flat frozen sample. This process ensured the integrity of the thickness of different regions of the skin. The skin discs were then cryosectioned (Thermo Scientific™ CryoStar™ NX70; Reinach, Switzerland) to obtain 50-μm thick sections starting from stratum corneum down to a skin depth of 400 μm. These lamellae enabled the amounts of TAK-441 to be determined as a function of position in the skin, encompassing the stratum corneum, epidermis, and upper dermis, respectively. Each lamella and the remaining dermis were individually extracted in 250-μL methanol for 4 h and TAK-441 was quantified by UHPLC-MS/MS.

### 1.4.6 Statistical Analysis

Data were expressed as the mean±SD. Outliers determined using the Grubbs test were discarded. Results were evaluated statistically using analysis of variance (ANOVA) one-way followed by the Tukey test for multiple comparisons or Student's t test. The level of significance was fixed at α=0.05.

### 1.5. Results and Discussion

### 1.5.1 Micelle Formulation Development and Characterization

A set of formulations (A-H) were prepared with constant TPGS content (10 mg/mL) but different target TAK-441 loadings: 100, 150, 200, 250, 300, 350, 400 and 500 mg of TAK-441 per g of TPGS. The drug loadings, drug contents, and incorporation efficiencies obtained for each formulation are given in Table 2. The highest drug content was provided by Formulation E (2.97±0.071 mg/mL).

TABLE 2
Micelle formulation characterization with respect to drug content and size.

| | | Target | Drug content | | | | | | |
|---|---|---|---|---|---|---|---|---|---|
| | TPGS | loading | Drug loading ± | Drug content ± | Entrapment | Size | | | |
| | content | (mg_{TAK-441}/ | SD (mg_{TAK-441}/ | SD (mg_{TAK-441}/ | efficiency ± | Zₐᵥ | | dᵥ | dₙ |
| # | (mg/mL) | g_{TPGS}) | g_{TPGS}) | mL_{formulation}) | SD (%) | (nm) | P.D.I. | (nm) | (nm) |
| A | 10 | 100 | 98.41 ± 1.87 | 0.98 ± 0.018 | 98.41 ± 1.87 | 14.01 | 0.063 | 12.29 | 10.45 |
| B | 10 | 150 | 147.21 ± 2.29 | 1.47 ± 0.023 | 98.14 ± 1.53 | 14.45 | 0.176 | 11.72 | 9.71 |
| C | 10 | 200 | 199.22 ± 3.86 | 1.99 ± 0.038 | 99.61 ± 1.93 | 13.83 | 0.071 | 11.79 | 9.39 |
| D | 10 | 250 | 239.5 ± 6.78 | 2.39 ± 0.068 | 95.80 ± 2.71 | 14.24 | 0.172 | 11.78 | 9.76 |
| E | 10 | 300 | 297.18 ± 7.08 | 2.97 ± 0.071 | 99.06 ± 2.36 | 14.33 | 0.172 | 11.32 | 9.86 |
| F | 10 | 350 | 216.34 ± 31.95 | 2.16 ± 0.320 | 61.81 ± 9.13 | 12.14 | 0.309 | 10.55 | 8.74 |
| G | 10 | 400 | 197.40 ± 28.56 | 1.97 ± 0.286 | 49.35 ± 7.14 | 16.92 | 0.306 | 12.07 | 10.51 |
| H | 10 | 500 | 207.35 ± 28.45 | 2.07 ± 0.285 | 41.47 ± 5.69 | 16.90 | 0.305 | 11.86 | 9.91 |

### 1.5.1.1 Size Characterization

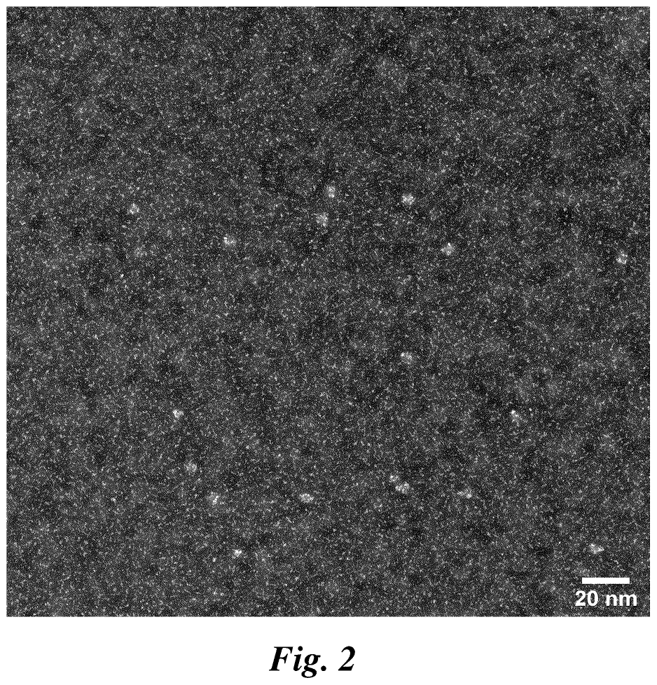

TAK-441 loaded TPGS micelles were characterized to determine their size using DLS (Table 2). All TAK-441 loaded micelle formulations presented uniform nanometer sizes with hydrodynamic diameters (Zₐᵥ) from 12.14 to 16.92 nm. The volume weighted diameter (dᵥ) measurements ranged from 10.55 to 12.29 nm and the number weighted diameter (dₙ) ranged from 8.74 nm to 10.51 nm. The TEM micrograph of the optimized formulation (Formulation E) is shown in FIG. 2 where it is apparent that micelles were spherical in shape with diameters ranging from 10 nm to 15 nm; these dimensions were confirmed by DLS (Table 2).

### 1.5.1.2 Development of HPC Micelle Gel

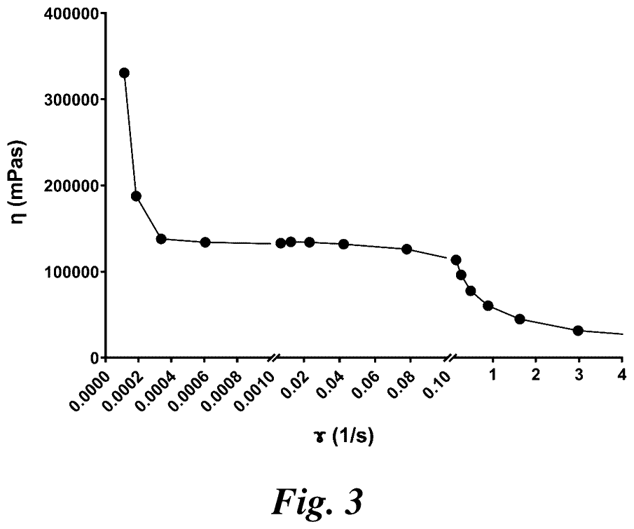

Formulation E was used to prepare a 3% HPC gel having a final drug content of 2.88 mg_{TAK-441}/g of gel formulation. The viscosity of the gel was be 283.5 Pas at a shear rate of 0.01 s⁻¹ (FIG. 3). As mentioned above, a 3% HPC gel with TAK-441 was used as the control—this would ensure that any superiority in skin delivery of TAK-441 from the micelle gel would be specifically due to the effect of the micelles.

### 1.5.2 Evaluation of TAK-441 Delivery In Vitro

1.5.2.1 Cutaneous Delivery of TAK-441 from Micelle Solution in Porcine Skin

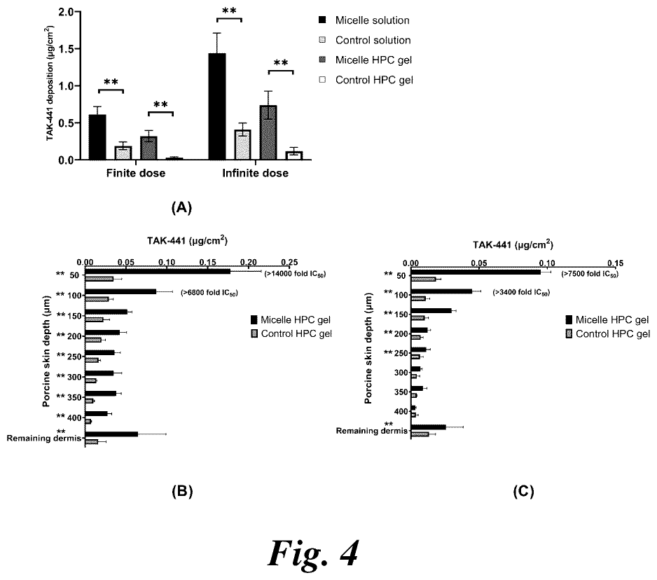

This study was performed to compare the cutaneous deposition and transdermal permeation of TAK-441 from the TPGS micelle solution and a control formulation. In these initial experiments, porcine skin was used to investigate TAK-441 delivery as it is one of the best surrogates for human skin (Dick and Scott, 1992; Schmook et al., 2001; Herkenne et al., 2006; Jacobi et al., 2007). The concentration of TAK-441 present in the receiver compartment was below the LOD of the UHPLC-MS/MS method—corresponding to a cumulative permeation of <0.1 pg/cm² after formulation application for 12 h. As shown in FIG. 4A, higher skin deposition was found in micelle solution groups (infinite and finite dose) compared to the control formulation. The amount of TAK-441 deposited in porcine skin from micelle solution and control formulation under infinite dose conditions was 1.44±0.27 μg/cm² and 0.41±0.09 μg/cm² (p=0.015, one-way ANOVA; n=6) and that for finite dose was found to be 0.61±0.11 μg/cm² and 0.19±0.052 μg/cm² (p=0.029, one-way ANOVA; n=6), respectively. Under infinite dose conditions, the concentration corresponding to the total amount of TAK-441 deposited in the whole skin sample after application of the micelle solution was greater than 7,100-fold higher than its IC₅₀ of 4.4 nM (Ohashi et al., 2012), and that at finite dose was greater than 3,000-fold higher.

1.5.2.2 Cutaneous Delivery of TAK-441 from Micelle-HPC Gel to Porcine Skin

The amount of TAK-441 deposited in porcine skin from the micelle HPC gel and control HPC gel formulation under infinite dose conditions was 0.74±0.19 μg/cm² and 0.12±0.05 μg/cm² (p=0.002, one-way ANOVA; n=6) and that for finite dose was found to be 0.32±0.08 μg/cm² and 0.03±0.01 μg/cm² (p=0.002, one-way ANOVA; n=6), respectively. The concentration of TAK-441 quantified in skin samples after 12 h of delivery was higher in micelle treated groups (solution and gel) as compared to control formulations in porcine skin.

The biodistribution studies enabled determination of the amounts of TAK-441 deposited as a function of depth. Biodistribution at infinite and finite dose revealed that greater amounts of TAK-441 were predominantly present at the target site, i.e., the epidermal region (FIG. 4B and FIG. 4C). Given the amounts of TAK-441 present in these smaller skin volumes, the estimated concentrations, were higher than those estimated for the skin sample as a whole. Hence, in the first lamella, going from 0 μm to 50 μm, the TAK-441 concentration achieved after application of the micelle-HPC gel at infinite and finite dose was greater than 14,000-fold and greater than 7,500-fold higher, respectively, than the IC₅₀; whereas, for the 50-100 μm region, the corresponding values were greater than 6,800-fold and greater than 3,400-fold higher, respectively.

### 1.5.2.3 Cutaneous Delivery of TAK-441 Using Micelle-HPMC Gel in Human Skin

### 1.5.2.3.1 Development of a Micelle-HPMC Gel

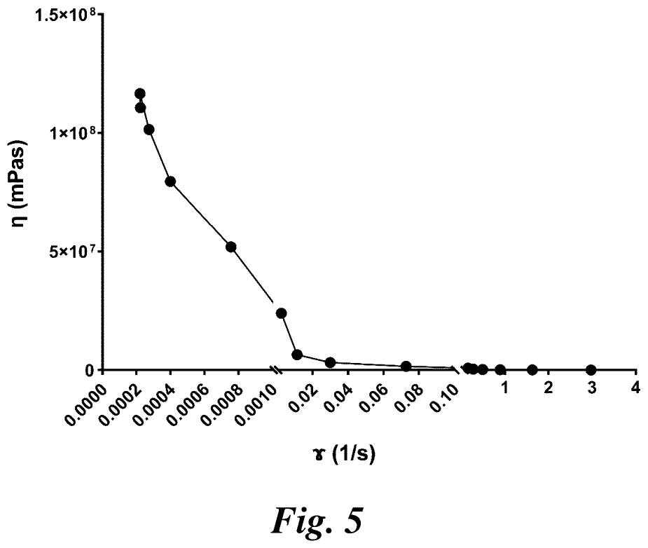

After the promising results in porcine skin, it was decided to perform delivery studies using a micelle gel formulation of TAK-441 and human skin. Since HPC precipitates at relatively low temperatures (cloud point: approximately 39° C.), however, and given that this characteristic could cause stability problems (Greiderer et al., 2011), it was decided to develop a HPMC-based micelle gel formulation (Table 3). The drug content was 2.5 mg_{TAK-441}/g of gel formulation. The viscosity of the gel was found to be 646.9 Pas at a sheer rate of 0.01 s⁻¹ (FIG. 5) with shear-thinning behavior that would be advantageous for the ease of application due to its better spreadability (Brummer and Godersky, 1999; Kwak et al., 2015).

TABLE 3
TAK-441-TPGS micelle-based HPMC gel composition

| | Concentration | |
|---|---|---|
| Component | (% w/w) | Role |
| TAK-441 | 0.25 | Investigational |
| | | molecule |
| TPGS | 0.93 | Micelle forming agent |
| HPMC | 5 | Gel base |
| Methocel ™ E5 Premium LV | 3 | Gel base |
| Glycerol | 2 | Rheology modifier |
| Sodium metabisulfite | 0.1 | Preservative |
| Water | Q.S. | Vehicle |

1.5.2.3.2 Determining Cutaneous Delivery and Biodistribution of TAK-441 from Micelle-HPMC Gel in Human Skin

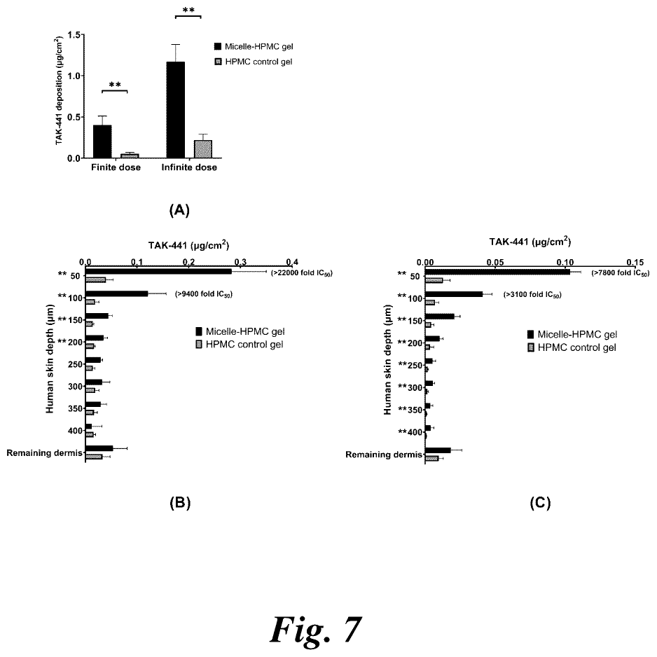

As observed for the porcine skin experiments, the amounts of TAK-441 permeated across human skin were again below the LOD of the UHPLC-MS/MS method after an application time of 12 h. Greater skin deposition of TAK-441 was observed for the micelle-HPMC gel (FIG. 7A). The amounts deposited from the micelle-HPMC gel and control HPMC gel formulations under infinite dose conditions were 1.17±0.21 μg/cm² and 0.22±0.07 μg/cm² (p=0.002, one-way ANOVA; n=6) and for finite dosing were 0.40±0.11 μg/cm² and 0.05±0.02 μg/cm² (p=0.002, one-way ANOVA; n=6), respectively.

Biodistribution at infinite and finite doses revealed similar profiles to those observed in porcine skin. Greater amounts of TAK-441 were again present in the epidermal region (FIG. 7B and FIG. 7C). For example, the estimated TAK-441 concentrations achieved on the 0-50 μm region, based on the amounts of TAK-441 delivered by micelle-HPMC gel at infinite and finite dose, were greater than 22,000-fold and greater than 7,800-fold higher than the IC₅₀, respectively; whereas, for the 50-100 μm region, the corresponding values were greater than 9,400-fold and greater than 3,100-fold higher, respectively.

The micelle-HPMC gel showed superiority over the HPMC control gel in all of the cutaneous delivery experiments performed. TAK-441 distribution in the HPMC gel is most likely more uniform than is the case for the HPMC control gel. TPGS micelles containing the TAK-441 in the lipophilic interior of the micelle, create a drug depot at the skin surface and, in particular, promote an accumulation of TAK-441 in the inter-cluster regions and the hair follicles (Kandekar et al., 2018; Lapteva et al., 2015, 2014b). The TPGS micelles will most likely disaggregate upon coming into contact with the lipophilic stratum corneum, thereby releasing the solubilized TAK-441 and making it available as a molecular dispersion.

Given its poor aqueous solubility, this availability will result in local (super) saturation of TAK-441. The high thermodynamic activity favors partitioning into the skin from the aqueous environment of the formulation (Hadgraft, 1999; Moser et al., 2001; Schwarb et al., 1999). The increased concentration of TAK-441 present in the stratum corneum results in an increased concentration gradient across the transport-limiting membrane and hence an increased flux. This increased flux is manifested at a macroscopic level by the greater amounts of TAK-441 measured at each skin depth in the cutaneous biodistribution profile.

Another important factor is water evaporation from the formulation on the skin surface. This factor will be more important under finite dose conditions and will contribute to the formation of a supersaturated solution of TAK-441 (Cilurzo et al., 2015). Since nanocarriers have been shown to accumulate in and around hair follicles, the follicular pathway may play an enhanced role in the cutaneous penetration of drugs applied using such delivery systems (Kandekar et al., 2018; Lapteva et al., 2015; Papakostas et al., 2011). It is also conceivable that the surfactant in the micelle formulation can act as a penetration enhancer; indeed, we have shown using MS imaging that TPGS can penetrate into the epidermis (Quartier et al., 2021a, 2021b).

### 1.6 Conclusion

The results confirmed that incorporation of TAK-441 in TPGS micelles was feasible and that the micelle-HPMC gel formulation of TAK-441 enabled its cutaneous delivery with the attainment of concentrations in the epidermal region that were orders of magnitude greater than the IC₅₀ for inhibition of the HH pathway. Given that one of the factors limiting the use of HH inhibitors generally, and constraining the clinical development of TAK-441, is the incidence of adverse effects, it was important to note that the concentrations in the permeation samples were substantially lower than the LOD of the sensitive UHPLC-MS/MS method (<1.29 ng/mL corresponding to a cumulative permeation <0.1 pg/cm²). This minimal permeation of TAK-441 across the skin should contribute to a reduction in the incidence of systemic side effects. It is clear that cutaneous delivery of TAK-441 from micelles to diseased human skin might be different to that observed in healthy tissue and dependent upon the type of the lesion and this would require further investigation in vivo.

### 1.7 Abbreviations

AE Adverse effects
BCC Basal cell carcinoma
BSA Bovine serum albumin
CAMP Cyclic adenosine monophosphate
DPBS Dulbecco's phosphate-buffered saline
GLI Glioma-associated oncogenes homologue
GPR161 G-Protein coupled receptor 161
HH Hedgehog
HPC Hydroxypropyl cellulose
HPMC Hydroxypropylmethylcellulose
KIF7 Kinesin Family Member 7 Protein
LOD Limit of detection
LOQ Limit of quantification
MFD Maximum feasible dose
NR Nile red
p53 Tumor suppressor protein p53
PC Primary cilium
P.D.I. Polydispersity index
PKA Protein kinase A
PTCH1 Patched 1 Protein
SMO Smoothened receptor
SUFU Suppressor of Fused homolog
TEM Transmission electron microscopy
TPGS D-α-Tocopherol polyethylene glycol 1000 succinate
UHPLC Ultra-high performance liquid chromatography
UV Ultraviolet

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
Aggarwal, N., Goindi, S., Mehta, S. D., 2012. Preparation and Evaluation of Dermal Delivery System of Griseofulvin Containing Vitamin E-TPGS as Penetration Enhancer. AAPS PharmSciTech 13, 67-74.
Athar, M., Tang, X., Lee, J. L., Kopelovich, L., Kim, A. L., 2006. Hedgehog signalling in skin development and cancer. Exp. Dermatol. 15, 667-677.
Bachhav, Y. G., Mondon, K., Kalia, Y. N., Gurny, R., Möller, M., 2011. Novel micelle formulations to increase cutaneous bioavailability of azole antifungals. J. Controlled Release 153, 126-132.
Brummer, R., Godersky, S., 1999. Rheological studies to objectify sensations occurring when cosmetic emulsions are applied to the skin. Colloids Surf. Physicochem. Eng. Asp. 152, 89-94.
Burness, C. B., 2015. Sonidegib: First Global Approval. Drugs 75, 1559-1566.
Cilurzo, F., Casiraghi, A., Selmin, F., Minghetti, P., 2015. Supersaturation as a tool for skin penetration enhancement. Curr. Pharm. Des. 21, 2733-2744.
Couvé-Privat, S., Bouadjar, B., Avril, M. F., Sarasin, A., Daya-Grosjean, L., 2002. Significantly high levels of ultraviolet-specific mutations in the smoothened gene in basal cell carcinomas from DNA repair-deficient xeroderma pigmentosum patients. Cancer Res. 62, 7186-7189.
Dahmana, N., Mugnier, T., Gabriel, D., Favez, T., Kowalczuk, L., Behar-Cohen, F., Gurny, R., Kalia, Y. N., 2021. Polymeric micelle mediated follicular delivery of spironolactone: Targeting the mineralocorticoid receptor to prevent glucocorticoid-induced activation and delayed cutaneous wound healing. Int. J. Pharm. 604, 120773.
Danial, C., Sarin, K. Y., Oro, A. E., Chang, A. L. S., 2016. An Investigator-Initiated Open-Label Trial of Sonidegib in Advanced Basal Cell Carcinoma Patients Resistant to Vismodegib. Clin. Cancer Res. Off. J. Am. Assoc. Cancer Res. 22, 1325-1329.
Daya-Grosjean, L., Sarasin, A., 2000. UV-specific mutations of the human patched gene in basal cell carcinomas from normal individuals and xeroderma pigmentosum patients. Mutat. Res. Mol. Mech. Mutagen. 450, 193-199.
Dick, I. P., Scott, R. C., 1992. Pig ear skin as an in-vitro model for human skin permeability. J. Pharm. Pharmacol. 44, 640-645.
Dlugosz, A., Agrawal, S., Kirkpatrick, P., 2012. Vismodegib. Nat. Rev. Drug Discov. 11, 437-438.
Gailani, M. R., Bale, A. E., 1997. Developmental Genes and Cancer: Role of Patched in Basal Cell Carcinoma of the Skin. JNCI J. Natl. Cancer Inst. 89, 1103-1109.
Goldman, J., Eckhardt, S. G., Borad, M. J., Curtis, K. K., Hidalgo, M., Calvo, E., Ryan, D. P., Wirth, L. J., Parikh, A., Partyka, J., Faessel, H., Gangolli, E., Stewart, S., Rosen, L. S., Bowles, D. W., 2015. Phase I Dose-Escalation Trial of the Oral Investigational Hedgehog Signaling Pathway Inhibitor TAK-441 in Patients with Advanced Solid Tumors. Clin. Cancer Res. 21, 1002-1009.
Gou, S., Monod, M., Salomon, D., Kalia, Y. N., 2022. Simultaneous Delivery of Econazole, Terbinafine and Amorolfine with Improved Cutaneous Bioavailability: A Novel Micelle-Based Antifungal “Tri-Therapy.” Pharmaceutics 14, 271.
Gould, S. E., Low, J. A., Marsters, J. C., Robarge, K., Rubin, L. L., de Sauvage, F. J., Sutherlin, D. P., Wong, H., Yauch, R. L., 2014. Discovery and preclinical development of vismodegib. Expert Opin. Drug Discov. 9, 969-984.
Greiderer, A., Steeneken, L., Aalbers, T., Vivó-Truyols, G., Schoenmakers, P., 2011. Characterization of hydroxypropylmethylcellulose (HPMC) using comprehensive two-dimensional liquid chromatography. J. Chromatogr. A 1218, 5787-5793.
Hadgraft, J., 1999. Passive enhancement strategies in topical and transdermal drug delivery. Int. J. Pharm. 184, 1-6.
Herkenne, C., Naik, A., Kalia, Y. N., Hadgraft, J., Guy, R. H., 2006. Pig ear skin ex vivo as a model for in vivo dermatopharmacokinetic studies in man. Pharm. Res. 23, 1850-1856.
Ishii, T., Shimizu, Y., Nakashima, K., Kondo, S., Ogawa, K., Sasaki, S., Matsui, H., 2014. Inhibition mechanism exploration of investigational drug TAK-441 as inhibitor against Vismodegib-resistant Smoothened mutant. Eur. J. Pharmacol. 723, 305-313.
Jacobi, U., Kaiser, M., Toll, R., Mangelsdorf, S., Audring, H., Otberg, N., Sterry, W., Lademann, J., 2007. Porcine ear skin: an in vitro model for human skin. Skin Res. Technol. 13, 19-24.
Jain, S., Song, R., Xie, J., 2017. Sonidegib: mechanism of action, pharmacology, and clinical utility for advanced basal cell carcinomas. OncoTargets Ther. Volume 10, 1645-1653.
Kandekar, S. G., del Río-Sancho, S., Lapteva, M., Kalia, Y. N., 2018. Selective delivery of adapalene to the human hair follicle under finite dose conditions using polymeric micelle nanocarriers. Nanoscale 10, 1099-1110.
Kandekar, S. G., Singhal, M., Sonaje, K. B., Kalia, Y. N., 2019. Polymeric micelle nanocarriers for targeted epidermal delivery of the hedgehog pathway inhibitor vismodegib: formulation development and cutaneous biodistribution in human skin. Expert Opin. Drug Deliv. 16, 667-674.
Kwak, M.-S., Ahn, H.-J., Song, K.-W., 2015. Rheological investigation of body cream and body lotion in actual application conditions. Korea-Aust. Rheol. J. 27, 241-251.
Lapteva, M., Mignot, M., Mondon, K., Möller, M., Gurny, R., Kalia, Y. N., 2019. Self-assembled mPEG-hexPLA polymeric nanocarriers for the targeted cutaneous delivery of imiquimod. Eur. J. Pharm. Biopharm. Off. J. Arbeitsgemeinschaft Pharm. Verfahrenstechnik EV 142, 553-562.
Lapteva, M., Möller, M., Gurny, R., Kalia, Y. N., 2015. Self-assembled polymeric nanocarriers for the targeted delivery of retinoic acid to the hair follicle. Nanoscale 7, 18651-18662.
Lapteva, M., Mondon, K., Möller, M., Gurny, R., Kalia, Y. N., 2014a. Polymeric Micelle Nanocarriers for the Cutaneous Delivery of Tacrolimus: A Targeted Approach for the Treatment of Psoriasis. Mol. Pharm. 11, 2989-3001.
Lapteva, M., Santer, V., Mondon, K., Patmanidis, I., Chiriano, G., Scapozza, L., Gurny, R., Möller, M., Kalia, Y. N., 2014b. Targeted cutaneous delivery of ciclosporin A using micellar nanocarriers and the possible role of inter-cluster regions as molecular transport pathways. J. Controlled Release 196, 9-18.
Lavasanifar, A., Samuel, J., Kwon, G. S., 2002. Poly(ethylene oxide)-block-poly(l-amino acid) micelles for drug delivery. Adv. Drug Deliv. Rev. 54, 169-190.
Madan, V., Lear, J. T., Szeimies, R.-M., 2010. Non-melanoma skin cancer 375, 13.
Moser, K., Kriwet, K., Froehlich, C., Kalia, Y. N., Guy, R. H., 2001. Supersaturation: enhancement of skin penetration and permeation of a lipophilic drug. Pharm. Res. 18, 1006-1011.
Nguyen, N. M., Cho, J., 2022. Hedgehog Pathway Inhibitors as Targeted Cancer Therapy and Strategies to Overcome Drug Resistance. Int. J. Mol. Sci. 23, 1733.
Ohashi, T., Oguro, Y., Tanaka, T., Shiokawa, Z., Tanaka, Y., Shibata, S., Sato, Y., Yamakawa, H., Hattori, H., Yamamoto, Y., Kondo, S., Miyamoto, M., Nishihara, M., Ishimura, Y., Tojo, H., Baba, A., Sasaki, S., 2012. Discovery of the investigational drug TAK-441, a pyrrolo[3,2-c]pyridine derivative, as a highly potent and orally active hedgehog signaling inhibitor: Modification of the core skeleton for improved solubility. Bioorg. Med. Chem. 20, 5507-5517.
Papakostas, D., Rancan, F., Sterry, W., Blume-Peytavi, U., Vogt, A., 2011. Nanoparticles in dermatology. Arch. Dermatol. Res. 303, 533-550.
Papas, A. M., 2021. Chapter 59-Vitamin E TPGS and its applications in nutraceuticals, in: Gupta, R. C., Lall, R., Srivastava, A. (Eds.), Nutraceuticals (Second Edition). Academic Press, pp. 991-1010.
Quartier, J., Lapteva, M., Boulaguiem, Y., Guerrier, S., Kalia, Y. N., 2021a. Polymeric micelle formulations for the cutaneous delivery of sirolimus: A new approach for the treatment of facial angiofibromas in tuberous sclerosis complex. Int. J. Pharm. 604, 120736.
Quartier, J., Rao, W., Slade, S., Métral, F., Lapteva, M., Kalia, Y. N., 2021b. DESI-MS imaging to visualize spatial distribution of xenobiotics and endogenous lipids in the skin. Int. J. Pharm. 607, 120967.
Robarge, K. D., Brunton, S. A., Castanedo, G. M., Cui, Y., Dina, M. S., Goldsmith, R., Gould, S. E., Guichert, O., Gunzner, J. L., Halladay, J., Jia, W., Khojasteh, C., Koehler, M. F. T., Kotkow, K., La, H., LaLonde, R. L., Lau, K., Lee, L., Marshall, D., Marsters, J. C., Murray, L. J., Qian, C., Rubin, L. L., Salphati, L., Stanley, M. S., Stibbard, J. H. A., Sutherlin, D. P., Ubhayaker, S., Wang, S., Wong, S., Xie, M., 2009. GDC-0449—A potent inhibitor of the hedgehog pathway. Bioorg. Med. Chem. Lett. 19, 5576-5581.
Roewert-Huber, J., Lange-Asschenfeldt, B., Stockfleth, E., Kerl, H., 2007. Epidemiology and aetiology of basal cell carcinoma: Epidemiology and aetiology of BCC. Br. J. Dermatol. 157, 47-51.
Samarasinghe, V., Madan, V., 2012. Nonmelanoma skin cancer. J. Cutan. Aesthetic Surg. 5, 3.
Schmook, F. P., Meingassner, J. G., Billich, A., 2001. Comparison of human skin or epidermis models with human and animal skin in in-vitro percutaneous absorption. Int. J. Pharm. 215, 51-56.
Schwarb, F. P., Imanidis, G., Smith, E. W., Haigh, J. M., Surber, C., 1999. Effect of concentration and degree of saturation of topical fluocinonide formulations on in vitro membrane transport and in vivo availability on human skin. Pharm. Res. 16, 909-915.
Staples, M. P., Elwood, M., Burton, R. C., Williams, J. L., Marks, R., Giles, G. G., 2006. Non-melanoma skin cancer in Australia: the 2002 national survey and trends since 1985. Med. J. Aust. 184, 6-10.
Vadlapudi, A. D., Cholkar, K., Vadlapatla, R. K., Mitra, A. K., 2014. Aqueous Nanomicellar Formulation for Topical Delivery of Biotinylated Lipid Prodrug of Acyclovir: Formulation Development and Ocular Biocompatibility. J. Ocul. Pharmacol. Ther. 30, 49-58.
Yauch, R. L., Dijkgraaf, G. J. P., Alicke, B., Januario, T., Ahn, C. P., Holcomb, T., Pujara, K., Stinson, J., Callahan, C. A., Tang, T., Bazan, J. F., Kan, Z., Seshagiri, S., Hann, C. L., Gould, S. E., Low, J. A., Rudin, C. M., de Sauvage, F. J., 2009. Smoothened Mutation Confers Resistance to a Hedgehog Pathway Inhibitor in Medulloblastoma. Science 326, 572-574.
Zhang, Z., Chu, Q., Wu, Y., Zhuang, X., Bao, Y., Wu, T., Tan, S., 2015. D-&alpha; -tocopherol polyethylene glycol succinate-based derivative nanoparticles as a novel carrier for paclitaxel delivery. Int. J. Nanomedicine 5219.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A composition comprising a hedgehog pathway inhibitor and a polymeric surfactant.

2. The composition of claim 1, wherein the hedgehog pathway inhibitor is active against vismodegib-resistant Smoothened receptor D473H mutant.

3. The composition of claim 1, wherein the hedgehog pathway inhibitor is selected from TAK-441, Vismodegib, Saridegib/Patidegib, Glasdegib, Sonidegib, Taladegib (Env-101), and BMS-833923 (XL-139).

4. The composition of claim 1, wherein the polymeric surfactant is biocompatible and/or biodegradable.

5. The composition of claim 1, wherein the polymeric surfactant is selected from D-α-Tocopherol polyethylene glycol 1000 succinate (TPGS), mPEG-dihex-PLA, a poloxamer, poly(e-caprolactone), a poly(L-amino acid), and polyvalerolactone.

6. The composition of claim 1, wherein the composition comprising a hedgehog pathway inhibitor and a polymeric surfactant comprises a micelle composition.

7. The composition of claim 6, wherein the micelle composition comprises spherical micelles having a diameter with a range from about 10 nm to about 100 nm.

8. The composition of claim 1, comprising a concentration of TPGS having a range from about 5 mg/mL to about 300 mg/mL.

9. The composition of claim 1, comprising a composition of TAK-441 having a range selected from about 100 mg to about 500 mg of TAK-441 per gram of TPGS and about 100 mg to about 300 mg of TAK-441 per gram of TPGS.

10. (canceled)

11. The composition of claim 1, further comprising a hydrogel.

12. The composition of claim 11, wherein the hydrogel is selected from hydroxypropylcellulose (HPC), hydroxypropyl methylcellulose (HPMC), a cellulose-based gel forming agent, and a poloxamer-based gelling agent.

13. The composition of claim 12, wherein the HPMC is selected from:
(a) an HPMC having a molecular mass of about 26 kDa, a methoxyl content having a range between about 19% to about 24%, and a hydroxypropoxyl content having a range between about 7% to about 12%;
(b) an HPMC having a molecular mass of about 10 kDa, a methoxyl content having a range between about 28% to about 30%, and a hydroxypropoxyl content between about 7% to about 12%; and
(c) combinations thereof.

14. The composition of claim 11, comprising:
about 0.25% (w/w) TAK-441;
about 0.93% (w/w) TPGS;
about 5% (w/w) of an HPMC having a molecular mass of about 26 kDa, a methoxyl content having a range between about 19% to about 24%, and a hydroxypropoxyl content having a range between about 7% to about 12%; and
about 3% of an HPMC having a molecular mass of about 10 kDa, a methoxyl content having a range between about 28% to about 30%, and a hydroxypropoxyl content between about 7% to about 12%.

15. The composition of claim 11, further comprising a rheology modifier.

16. The composition of claim 15, wherein the rheology modifier is selected from glycerol, a low and high molecular weight cellulose, and a sorbitol.

17. The composition of claim 11, further comprising a preservative.

18. The composition of claim 17, wherein the preservative is selected from sodium metabisulfite, benzyl alcohol, benzalkonium chloride, chlorobutanol, sodium benzoate, potassium sorbate, methylparaben and propylparaben.

19. The composition of claim 1, wherein the composition retains between about 90% to 100% hedgehog pathway inhibitor content after storage for about 6 months.

20. A method for treating a disease, disorder, or condition associated with a hedgehog (HH) signaling pathway, the method comprising administering to a subject in need of treatment thereof, the composition of claim 1.

21. The method of claim 20, wherein the composition is:
(a) administered topically;
(b) delivered cutaneously;
(c) delivered to a viable epidermis of the subject; or
(d) delivered to an upper dermis of the subject.

22.-24. (canceled)

25. The method of claim 21, wherein the topical administration results in negligible transdermal permeation.

26. The method of claim 20, wherein the disease, disorder, or condition associated with a hedgehog (HH) signaling pathway:
(a) comprises a skin disease, disorder, or condition;
(b) involves a mutation in a Patched 1 (PTCH1) protein; and/or
(c) involves a mutation in a smoothened (SMO) protein.

27. The method of claim 26, wherein the skin disease, disorder, or condition comprises a skin cancer.

28. The method of claim 27, wherein the skin cancer comprises basal cell carcinoma.

29. The method of claim 28, wherein the subject has or is suspected of having locally advanced basal cell carcinoma and/or metastatic basal cell carcinoma.

30. (canceled)

31. The method of claim 29, wherein the subject is not eligible for surgical or radiotherapeutic treatment.

32. The method of claim 28, wherein administering the composition to the subject attenuates progression of the basal cell carcinoma.

33. The method of claim 28, wherein the basal cell carcinoma involves a vismodegib-resistant SMO mutant D473H.

34. The method of claim 20, wherein administering the composition to the subject prevents activation of the HH signaling pathway.

35. (canceled)

36. The method of claim 26, wherein:
(a) the mutation in the Patched 1 (PTCH1) protein leads to a loss of function of the PTCH1 protein;
(b) the mutation in the smoothened (SMO) protein involves a gain of function in the SMO protein; and/or
(c) the loss of function of the PTCH1 protein or the gain of function in the SMO protein leads to activation of one or more GLI transcription factors.

37.-39. (canceled)

40. The method of claim 36, wherein the activation of one or more GLI transcription factors results in hyperproliferation of basal cells associated with basal cell carcinoma.

41. The method of claim 20, wherein administering the composition to the subject inhibits SMO.

42. The method of claim 26, wherein the mutation in SMO results in resistance to treatment with the hedgehog pathway inhibitor.

43. The method of claim 42, wherein the resistance to treatment with the hedgehog pathway inhibitor involves a mutation in a drug binding site of SMO.

44. The method of claim 43, wherein the drug binding site of SMO is SMO-Q476 and/or SMO-D473.
